(19) United States
(12) Patent Application Publication
(10) Pub. No.: US 2010/0317578 A1
(43) Pub. Date: Dec. 16, 2010

(54) METHOD OF PROTECTING AGAINST STROKE THROUGH THE USE OF A DELTA OPIOID PEPTIDE

(76) Inventors: Cesario V. Borlongan, Odessa, FL (US); Tsung-Ping Su, Cockeysville, MD (US); Yun Wang, Ellicott City, MD (US)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 12/813,191

(22) Filed: Jun. 10, 2010

Related U.S. Application Data
(60) Provisional application No. 61/185,853, filed on Jun. 10, 2009.

(51) Int. Cl. A61K 38/08; A61P 25/00; A61P 9/10

(52) U.S. Cl. 514/8.4; 514/17.7

(57) ABSTRACT

The subject invention pertains to uses of delta opioid peptides and salts thereof for promoting neurogenesis and to pharmaceutical compositions containing such peptides and salts as active ingredients. Specifically exemplified herein is [_{D}-Ala²,_{D}-Leu⁵]enkephalin (DADLE) and salts thereof. The peptides of the present invention upregulate glial cell-derived neurotrophic factor (GDNF) in the nervous system and are useful for prevention and treatment of diseases and conditions associated with neurological injury, in particular, stroke.

### CROSS-REFERENCE TO RELATED APPLICATION

The subject application claims the benefit of U.S. Provisional Application Ser. No. 61/185,853, filed Jun. 10, 2009, which is incorporated herein by reference in its entirety, including all figures, tables, amino acid sequences, and nucleic acid sequences.

### GOVERNMENTAL SUPPORT

This invention was made with Government support awarded by the NIDA, NIH Intramural Funds. The Government has certain rights in the invention.

### FIELD OF INVENTION

The subject invention relates to uses of delta opioid peptides and salts thereof for preventing, treating or ameliorating diseases or disorders associated with neurological injury, in particular, stroke.

### BACKGROUND

Stroke is the third leading cause of death and adult disability in the United States. It is associated with an abrupt, unpredictable, and largely irreversible initial brain cell death, followed by a cascade of secondary progressive cell death (e.g. apoptosis and necrosis). This permanent neuronal injury results in severe motor and neurological deficits or even death. Abrogating, controlling, or reversing the cascade of cell death would prevent or delay the onset and progression of stroke, thereby reducing stroke-related morbidity and mortality. Therefore, therapeutic agents and compositions that protect against brain cell death and/or promote formation of new brain cells or neurogenesis are urgently needed.

Opioids are well-known analgesics [2] and are associated with drug abuse [1]. The drug activity of opioid peptides is mediated by opioid receptors including mu, kappa, and delta receptors. Opioid peptides may also play a significant role in various behavioral and physiological responses. It has been reported that morphine, a mu opioid, induces Fas-mediated cell death [9].

Delta opioid peptides, such as [_{D}-Ala²,_{D}-Leu⁵]enkephalin (DADLE), have been shown to induce hibernation in summer ground squirrels. They have also been found to enhance preservation and survival of isolated or transplanted lungs and hearts. It has been reported that hibernation-inducing delta opioid peptides, such as DADLE, also have anti-ischemic effects. These delta opioid peptides, however, have not been previously reported to play any role in neurogenesis.

### BRIEF SUMMARY

The present invention provides novel and advantageous therapeutic methods for treating diseases and conditions associated with neurological injury, in particular, stroke. The methods comprise administering to a subject in need of such treatment an effective amount of an isolated peptide or salt thereof. In a preferred embodiment, the peptide of the present invention is a delta opioid peptide: [_{D}-Ala²,_{D}-Leu⁵]enkephalin (DADLE), having an amino acid sequence of Tyr-_{D}-Ala-Gly-Phe-_{D}-Leu (SEQ ID NO:1).

Advantageously, the methods of the present invention can be used to promote neurogenesis in a subject. In addition, the methods of the present invention can be used to increase the expression of glial cell-derived neurotrophic factor (GDNF) in a subject.

The present invention also provides pharmaceutical compositions that promote neurogenesis, comprising an effective amount of the peptide and/or salt of the present invention as an active ingredient and a pharmaceutically-acceptable carrier or diluent.

The methods of the present invention are particularly useful for treating conditions associated with neurological injury including, for example, stroke, acute stroke, cerebral artery stroke, ischemic stroke, ischemic injury, acute ischemic injury, and cerebral infarction.

### BRIEF DESCRIPTION OF THE DRAWINGS

FIG. 1 shows that DADLE attenuates ischemia-induced asymmetrical motor deficits. Ischemic animals pretreated with saline (S), naltrexone alone (N) or naloxone methiodide alone (NM) displayed significant biased swing activity (Panel A), postural bias (Panel B), spontaneous rotational behavior (Panel C), and forelimb akinesia (Panel D). In contrast, ischemic animals pretreated with DADLE+naltrexone (D+N) or DADLE+naloxone methiodide (D+NM) exhibited significant dysfunctions in the same behavioral tests only at 0-24 hours post occlusion and reperfusion surgery of the middle cerebral artery (MCAor) (Days 0 and 1), and thereafter showed near-normal behaviors at 48 hours and 72 hours post-MCAor (Days 2 and 3). In contrast, ischemic animals pretreated with DADLE alone displayed near-normal behaviors throughout the post-MCAor test period.

FIGS. 2A-D show that DADLE reduces necrotic and apoptotic cell death associated with ischemia. FIG. 2A shows images of brain sections taken after MCAor, in which the animals were pretreated with saline, DADLE, DADLE+naloxone methiodide, or DADLE+naltrexone. Triphenyltetrazolium chloride (TTC) staining at 72 hours post-MCA or revealed that the striata from ischemic animals exposed to ischemia/reperfusion surgery and pretreated with saline showed dehydrogenase deficient tissue (negative TTC stains). In contrast, the striata from ischemic animals pretreated with DADLE, DADLE+naloxone methiodide, or DADLE+naltrexone did not reveal any dehydrogenase deficiency. FIG. 2B shows p53 mRNA expression in the intact striatum of normal controls and in the striatum of ischemic animals treated with saline versus DADLE after ischemia. Ischemic animals exposed to ischemia/reperfusion injury and treated with saline showed a significant increase in mRNA expression of p-53 in their ischemic striata (65% increment compared to intact striatum of normal, control animals) at 24 hours after stroke surgery. In contrast, ischemic animals treated with DADLE exhibited only a small increment in p-53 mRNA expression in their ischemic striata at the same time period (28% increment compared to intact striatum of normal, control animals; not significantly different from control values). Comparisons of ischemic striata between these two groups showed a marked reduction in p-53 mRNA expression in DADLE-treated ischemic animals compared to saline-treated ischemic animals. FIG. 2C shows immunohistochemical analysis for phenotypic markers of apoptosis. Immunohistochemical analyses of phenotypic markers of apoptosis revealed that DADLE significantly reduced caspase-3-(Panels a-d) and Fas-positive cells (Panels e-h) in DADLE-treated ischemic animals compared to saline-treated ischemic animals. Quantitative data shown in bar graphs and represent means±S.E.M. Asterisks correspond to statistical significance at p<0.05. (a), 100 μm (b). FIG. 2D shows necrotic and apoptotic cells in saline-treated ischemic animals. To better capture the necrotic and apoptotic cells in saline-treated ischemic animals, higher magnification images are generated from propidium iodide (Panel a) and caspase-3 (Panel b) immunofluorescently labeled striatal cells, respectively. Scale bar=50 μm.

FIG. 3 shows that DADLE increases expression of glial cell derived neurotrophic factor (GDNF), but not nerve growth factor (NGF), in brain tissues. ELISA assays revealed that the levels of GDNF proteins, but not NGF, were significantly higher in striatal and cortical tissues harvested from ischemic animals treated with DADLE compared to those treated with saline. Data are expressed as mean percent of control ±S.E.M. Asterisk indicates p<0.05. n=6 samples for each neurotrophic factor examined.

FIG. 4 is a schematic diagram showing the timeline of experimental procedures of the present invention.

### BRIEF DESCRIPTION OF THE SEQUENCES

SEQ ID NO:1 is an amino acid sequence useful according to the subject invention.

### DETAILED DESCRIPTION

The present invention pertains to novel uses as neurogenesis-promoting agents of delta opioid peptides and salts thereof. The peptides and compositions of the present invention are potent neuroprotective and neurogenesis-promoting agents, useful for preventing and/or treating diseases and conditions associated with neurological injury, in particular, stroke. In a preferred embodiment, the present invention pertains to the use of a delta opioid peptide: [_{D}-Ala²,_{D}-Leu⁵]enkephalin (DADLE), having an amino acid sequence of Tyr-_{D}-Ala-Gly-Phe-_{D}-Leu (SEQ ID NO:1).

The present invention is based, at least in part, on the surprising discovery that delta opioid peptides, such as [_{D}-Ala²,_{D}-Leu⁵]enkephalin (DADLE), promote neurogenesis. The peptides of the present invention significantly increase expression of glial cell-derived neurotrophic factor (GDNF), which plays a pivotal role in the generation, growth, differentiation, proliferation and/or survival of various types of neurons, in particular, dopaminergic neurons and motorneurons. In addition, the peptides of the present invention effectively protect neurons against apoptosis and necrosis, and markedly reduce levels of apoptotic/necrotic cell death markers such as p-53, caspase-3, and Fas. It is further contemplated that the peptides of the present invention exert their neuroprotective effects via both opioid and non-opioid pathways. Treatment with the peptides of the present invention effectively reduces cerebral infarction and ischemic injury, abrogates initial as well as secondary progressive cell death, and improves neurological, motor, and behavioral function of subjects suffering from neurological injury. Thus, the peptides of the present invention not only exert neuroprotective effects through induction of hibernation-like state (hypothermia), but also through enhancement of endogenous neurogenesis.

It has been recently reported that delta opioids may be involved in ischemia [3-5]. Following occlusion of the middle cerebral artery in mice, delta binding sites were decreased at least 6 hr earlier than reductions in mu or kappa binding sites, and concomitant with the extension of the infarct core [3]. The early reduction in delta receptor binding sites, prior to any observable brain damage, suggests that these receptors are very sensitive to brain insults. Thus, it is contemplated by the present inventors that stimulating the delta receptors would produce anti-ischemic effects.

A “natural hibernation” condition has been suggested to achieve anti-ischemic effects [4,5]. The preservation of isolated rat hearts can be improved by pharmacologically duplicating the common pathway to natural hibernation and ischemic preconditioning, that is, through an opening of the ATP-sensitive potassium channels [4]. Brain tissues collected from hibernating ground squirrels were more tolerant to hypoxia and aglycemia than those tissues from active squirrels [5]. Thus, it is contemplated by the present inventors that hibernation-inducing drugs, such as DADLE, could act as anti-ischemic agents as the mechanisms (e.g., oxidative stress, free radical formation) involved in the survival/degeneration of tissues in the peripheral nervous system and the central nervous system (CNS) share commonalities.

In the present invention, the protective effect of DADLE in the CNS has examined by pretreating young adult rats with DADLE and subsequently exposing them to occlusion and reperfusion surgery of the middle cerebral artery (MCAor). Routine behavioral tests were employed to characterize functional alterations during an acute post-ischemia period. The volume and size of infarction were analyzed during histological examinations using triphenyltetrazolium chloride [6] for determining dehydrogenase activity. In addition, as apoptotic mechanism of cell death accompanies the MCAor stroke model [7,8], the effects of DADLE on levels of p-53 mRNA, an apoptotic marker, were also examined. Finally, pharmacologic manipulations of DADLE activity, using delta opioid antagonists naltrexone (a universal opioid receptor blocker) and naloxone methaiodide (a peripheral opioid receptor blocker), were performed to study the mediation of DADLE action by opioid receptors in the CNS.

The results, as shown in the Examples, demonstrate that DADLE protected against MCAor ischemia-induced behavioral deficits. Animals pretreated with DADLE and subsequently exposed to the MCAor surgery did not show significant behavioral deficits as revealed by a battery of tests, whereas ischemic animals pretreated with saline alone exhibited significant dysfunctions in all behavioral tests. Delta opioid blockers naltrexone and naloxone methaiodide transiently antagonized the protective effects of DADLE at 24 hr post-MCAor but not thereafter, indicating that DADLE's effects are only partially mediated by opioid receptors. The data on TTC staining (a marker of irreversible cell damage) revealed almost no detectable dehydrogenase-deficient tissue (necrotic infarction) in the striatum (the ischemic core) at 24 hr or 72 hr post-MCAor in ischemic animals treated with DADLE alone or DADLE+opioid blockers. While opioid blockers transiently suppressed the behavioral protection by DADLE, they did not antagonize DADLE's inhibition of ischemia-induced necrosis.

In addition, striatal p-53 mRNA expression, an index of apoptosis, was significantly suppressed by DADLE at 24 hr post-MCAor. In contrast, an increment in striatal p-53 mRNA expression was noted in ischemic animals that received saline alone. Thus, pretreatment with DADLE was shown to rescue both ischemia-induced necrotic and apoptotic cell injury, which could have promoted the observed behavioral protection.

The observation that DADLE exerts therapeutic effects in a stroke model further reveals that the opioid system plays a pivotal role in cell degeneration and regeneration. The present invention demonstrates delta opioid peptides and agonists protected the brain from ischemic damage, including apoptosis and necrosis.

The significant reductions in cerebral infarction and apoptotic cell death markers (p-53 mRNA expression, caspase-3, and Fas) indicate that DADLE exerts its protective effects in the CNS. It is demonstrated by the present inventors that DADLE blocks and even reverses the loss of dopamine transporters induced by chronic methamphetamine treatment [12,13]. Moreover, DADLE inhibits accumulations of superoxide anions and hydroxyl radicals [14], which have been shown as exacerbating factors for many neurological disorders, including stroke [15,16].

It is further contemplated that ischemia-induced cell death is associated with dysfunction of the dopaminergic system, specifically the striatal dopamine pathway. The present inventors have reported that many functional deficits associated with the MCAor are related to striatal dopamine-mediated behaviors [17]. Animals with striatal ischemia display methamphetamine-induced abnormal rotational behaviors [18], as well as impairments in passive avoidance and Morris water maze tests [19]. Thus, the dopamine-rich innervated striatum is a critical brain area for developing neuroprotective therapeutics for prevention and/or treatment of stroke.

Surprisingly, it has now been discovered that DADLE has protective effects in the central nervous system and is capable of increasing expression of GDNF in the striatum and cortex, which are the brain areas targeted by MCAor stroke. It has been demonstrated by the present inventors that intracerebral infusion of GDNF protects against cerebral ischemia [6]. Moreover, it has been established that GDNF is a highly selective dopamine neuron survival agent [20,21]. The present inventors also recently reported that DADLE enhances embryonic dopamine cell viability in vitro and following intrastriatal transplantation [22], and protects adult dopaminergic neurons in vivo against 6-hydroxydopamine-induced neurotoxicity [23]. The present invention demonstrates that GDNF striatal levels are significantly increased following DADLE treatment, showing that the striatal dopaminergic system is a highly potent target for DADLE in reversing ischemia cell injury, as well as other diseases characterized by dopamine dysfunctions.

In addition, the differential onsets of behavioral recovery between ischemic animals that received DADLE and those treated with DADLE and opioid receptor blockers demonstrate that DADLE exerts its neuroprotective effects via both the opioid pathway and the non-opioid pathway. DADLE-treated ischemic animals displayed significantly reduced behavioral deficits in the battery of tests throughout the post-MCAor test period. In contrast, those ischemic animals that received a combination of DADLE and opioid receptor blockers did not exhibit improvements in stroke-induced behavioral deficits until 48 hours, and such deficits can persist up to 72 hours post-MCAor. This observation of transient behavioral abnormalities in ischemic animals treated with DADLE and opioid antagonists suggests that DADLE only partially exerted its protection via the opioid system, and non-opioid mechanisms appear to mediate the majority of protective effects of DADLE. Accordingly, blocking DADLE via the opioid pathway would only partially inhibit DADLE's therapeutic benefits.

The partial antagonistic effects of opioid blockers were reflected in the delayed onset of behavioral recovery in ischemic animals treated with DADLE and opioid blockers. Specifically, for the DADLE+opioid blocker treated ischemic animals that exhibited behavioral deficits at early post-MCAor phase, recovery from ischemic damages initiated at a later post-MCAor time point. Such delayed functional recovery is due to the temporal profile of DADLE's non-opioid therapeutic action (e.g., GDNF upregulation). Based on the reported neurotrophic effects of GDNF on endogenous cells [24,25], recruitment of newly formed cells from neurogenic sites (e.g., subventricular zone) towards the ischemic striatal area may be delayed, thereby corresponding to the delayed behavioral recovery. Recent data from our laboratory indicate DADLE robustly induces neurogenesis (data not shown).

Furthermore, DADLE effectively prevented stroke cell death cascades and demonstrated neuroprotective effects via both opioid and non-opioid pathways. Specifically, non-opioid therapeutic action of DADLE effectively abrogates ischemic penumbra in stroke subjects. The present behavioral and histological results showed evolution of the penumbra during the acute phase of ischemic stroke [26,27]. Stroke-induced histological alterations were also examined using TTC staining. TTC staining revealed that DADLE treated ischemic animals, as well as those that received combined DADLE and opioid blockers, all demonstrate near absent infarcts at 24 hours post-stroke (i.e., the earliest time point such infarct evaluation was conducted). Despite the absence of massive infarcts, ischemic animals treated with DADLE and opioid blockers initially exhibited significant behavioral deficits at 24 hours, then the abnormal behaviors eventually improved at 48 hours and remained near normal levels at 72 hours post-stroke. Thus, despite near normal gross histology, the behavioral deficits at very acute stage of stroke could be due to subtle lesions and/or apoptotic cell death not detected by TTC staining. The evolution of ischemic penumbra in these animals, which showed transient behavioral deficits, could have been similarly abrogated by DADLE's non-opioid therapeutic action.

In addition, DADLE-mediated GDNF upregulation can occur even prior to stroke, since DADLE was administered repeatedly starting at 6 hours pre-stroke. It is reported that optimal GDNF therapeutic effects are achieved when initiated at an ample time interval prior to injury [28,29]. Moreover, DADLE neutrophic effects, particularly in those animals co-treated with opioid blockers, can be achieved via a two-pronged pathway. One pathway is to combat directly the ischemic penumbra, and the other pathway is via recruitment of endogenous stem cells. The combination of these two neuroprotective mechanisms was more robust in DADLE treated ischemic animals, while the manifestation of neuroprotective effects was delayed (i.e., 48 hours) for those that received combined DADLE and opioid blockers.

### Treatment of Neurological Injury

The peptides and compositions of the present invention, through administration to a subject, are useful for preventing, treating or ameliorating diseases or conditions associated with neurological injury, in particular, stroke. Advantageously, delta opioid peptides, such as DADLE, promote neurogenesis and are useful for preventing neuronal cell death as well as promoting neuronal cell growth. The peptides of the present invention significantly upregulate GDNF, a neurotropic factor shown to prevent neuron apoptosis and necrosis, promote neuron regeneration, growth and survival, and stimulate the re-growth and repair of damaged neuronal cells. In addition, the peptides of the present invention effectively reduce levels of apoptotic/necrotic markers such as p53, caspase-3 and Fas.

The term “subject,” as used herein, describes an organism, including mammals such as primates, to which treatment with the compositions according to the present invention can be administered. Mammalian species that can benefit from the disclosed methods of treatment include, but are not limited to, apes, chimpanzees, orangutans, humans, monkeys; and other animals such as dogs, cats, horses, cattle, pigs, sheep, goats, chickens, mice, rats, guinea pigs, and hamsters. Typically, the subject is a human.

The term “treating,” as used herein, includes but is not limited to, reducing, suppressing, inhibiting, lessening, or affecting the progression, severity, and/or scope of a condition, chance of re-occurrence or returning of a disease after a remission.

The term “preventing,” as used herein, includes but is not limited to, delaying the onset of symptoms, preventing relapse to a disease, decreasing the number or frequency of relapse episodes, increasing latency between symptomatic episodes, or a combination thereof.

The term “neurogenesis,” as used herein, refers to generation of new neuronal cells from proliferating neural stem/progenitor cells, differentiation of neural stem/progenitor cells into new neural cell types, and/or migration and/or maturation of new neuronal cells.

In an embodiment, the present invention provides a method for preventing, treating or ameliorating a disease or condition associated with neurological damage. The method comprises administering, to a subject in need of such treatment (e.g. neuroprotection and neurogenesis), an effective amount of a delta opioid peptide or salt thereof. In an embodiment, the delta opioid peptide or salt thereof is administered to a human subject who has symptoms of, or is diagnosed with, stroke.

In an embodiment, the present invention prevents, treats or ameliorates a disease or condition associated with neuronal injury, damage, death or degeneration. In another embodiment, the present invention prevents, treats or ameliorates a disease or condition in which normal neuronal function is impaired, or protection or generation (re-generation) of neuronal cells, or modulation or repair neural function would be beneficial.

The term “an effective amount” or “therapeutically effective amount,” as used herein, refers to that an amount that is capable of preventing, treating or ameliorating a disease or condition or otherwise capable of producing an intended therapeutic effect. For instance, the effective amount of the peptides and compositions of the present invention is an amount capable of producing neuroprotective effects, promoting neurogenesis, and/or increasing expression of GDNF in a subject.

The present invention is particular useful for preventing or treating a disease or condition responsive to, at least in part, the activity of GDNF. In addition, the present invention is particular useful for preventing or treating a disease or condition associated with, at least in part, the dopaminergic pathway. In a specific embodiment, the present invention can be used to prevent, treat or ameliorate a disease or disorder, in which generation of motor neurons or dopaminergic neurons would be beneficial.

The peptides and compositions of the present invention can be used to prevent, treat or ameliorate diseases and conditions associated with neurological injury or neuronal death including, but not limited to, stroke, acute stroke, cerebral artery stroke, ischemic stroke, ataxia, dyskinesia, ischemic injury, acute ischemic injury, and cerebral infarction.

Specifically exemplified herein are uses of the peptides and compositions of the present invention for preventing, treating or ameliorating stroke, in particular ischemic stroke. In a specific embodiment, the present invention can be used to prevent, treat or ameliorate cerebral artery stroke, in particular middle cerebral artery stroke.

In another embodiment, the present invention is useful for preventing or delaying the onset of stroke, and/or treating or alleviating stroke symptoms associated with initial brain cell apotosis and necrosis. In another embodiment, the present invention is useful for preventing or delaying the progression of stroke, and/or treating or alleviating stroke symptoms associated with secondary progressive cell apotosis and necrosis.

In addition, the peptides and compositions of the present invention can be used to prevent, treat or ameliorate diseases and conditions associated with injury to the central and peripheral nervous systems, including but limited to, injury, lesion, or trauma to the brain, the spinal cord, and nerves. In a specific embodiment, the present invention can be used to prevent, treat, or ameliorate diseases and conditions associated with ischemic injury or cerebral ischemic neuronal damage.

In addition, the peptides and compositions of the present invention can be used to prevent, treat or ameliorate diseases and conditions associated with loss of neuronal cells or defects in neuronal function, including but not limited to, behavioral, motor, cognitive, sensory, and speech defects. In a specific embodiment, the present invention is useful for preventing, treating or ameliorating motor system disorders, such as stroke, and symptoms of atypical movement; rigidity or stiffness of the limbs and trunk; bradykinesia; postural bias or instability; impaired balance and coordination; muscular rigidity; and ataxia.

### Therapeutic Compositions and Formulations

The present invention further provides therapeutic compositions that contain a therapeutically effective amount of the peptides or salts and a pharmaceutically acceptable carrier or adjuvant. The present invention also contemplates prodrugs or metabolites of the peptides or salts thereof.

In one embodiment, acids suitable for preparing the peptide salts include, are but not limited to, hydrochloric acid, acetic acid, aspartic acid, citric acid, fumaric acid, hippuric acid, lactic acid, malic acid, phosphoric acid, sulfuric acid, succinic acid, carbonic acid, and gluconic acid.

Further, the therapeutic composition of the present invention can comprise the peptides or salts of the present invention as a first active ingredient, and one or more additional active ingredients comprising a second neuroprotective agent known in the art.

As used herein, the terms “pharmaceutically acceptable”, “physiologically tolerable” and grammatical variations thereof, include compositions, carriers, diluents and reagents, are used interchangeably and represent that the materials are capable of administration to or upon a subject such as mammal.

The term “prodrug,” as used herein, refers to a metabolic precursor of a peptide of the present invention or pharmaceutically acceptable form thereof. In general, a prodrug comprises a functional derivative of a peptide, which may be inactive when administered to a subject, but is readily convertible in vivo into an active metabolite peptide.

Conventional procedures for the selection and preparation of suitable prodrug derivatives are described, for example, in “Design of Prodrugs”, ed. H. Bundgaard, Elsevier, 1985. Preferably, a prodrug of the present invention enhances desirable qualities of the peptide of the present invention including, but not limited to, solubility, bioavailability, and stability. Hence, the peptides employed in the present methods may, if desired, be delivered in a prodrug form. Prodrugs of the peptides employed in the present invention may be prepared by modifying functional groups present in the peptide such that the modifications are cleaved, either in routine manipulation or in vivo, to the parent peptide/compound.

The term “metabolite,” refers to a pharmacologically active product, including for example, an active intermediate or an ultimate product, produced through in vivo metabolism of a peptide of the present invention in a subject. A metabolite may result, for example, from the anabolic and/or catabolic processes of the administered compound (e.g. peptide) in a subject, including but not limited to, the oxidation, reduction, hydrolysis, amidation, deamidation, esterification, deesterification, enzymatic cleavage, and the like.

The peptide salts of the present invention may be compounded, for example, with the usual non-toxic, pharmaceutically acceptable carriers for tablets, pellets, capsules, liposomes, suppositories, intranasal sprays, solutions, emulsions, suspensions, aerosols, targeted chemical delivery systems (Prokai-Tatrai, K.; Prokai, L; Bodor, N., J. Med. Chem. 39:4775-4782, 1991), and any other form suitable for use. The carriers which can be used are water, glucose, lactose, gum acacia, gelatin, mannitol, starch paste, magnesium trisilicate, corn starch, keratin, colloidal silica, potato starch, urea and other carriers suitable for use in manufacturing preparations, in solid, semisolid, liquid or aerosol form, and in addition auxiliary, stabilizing, thickening and coloring agents and perfumes may be used.

The present invention contemplates therapeutic compositions useful for practicing the therapeutic methods described herein. Therapeutic compositions of the present invention contain a physiologically tolerable carrier together with a therapeutically effective amount of a peptide as described herein, dissolved or dispersed therein as an active ingredient.

The peptides used in these therapies can also be in a variety of forms. These include for example, solid, semi-solid and liquid dosage forms, such as tablets, pills, powders, liquid solutions or suspensions, suppositories, injectable and infusible solutions. The preferred form depends on the intended mode of administration and therapeutic application.

The compositions also preferably include conventional pharmaceutically acceptable carriers and adjuvants which are known to those of skill in the art. Preferably, the compositions of the invention are in the form of a unit dose and will usually be administered to the patient one or more times a day.

The present peptides and compositions can be in a form that can be combined with a pharmaceutically acceptable carrier. In this context, the peptide or compound may be, for example, isolated or substantially pure. The term “carrier,” as used herein, includes a diluent, adjuvant, excipient, or vehicle with which the compound is administered. Such pharmaceutical carriers can be sterile liquids, such as water and oils, including those of petroleum oil such as mineral oil, vegetable oil such as peanut oil, soybean oil, and sesame oil, animal oil, or oil of synthetic origin. Saline solutions and aqueous dextrose and glycerol solutions can also be employed as liquid carriers, particularly for injectable solutions. Particularly preferred pharmaceutical carriers for treatment of or amelioration of neurological disorder in the central nervous system are carriers that can penetrate the blood/brain barrier.

The amount of active ingredient that may be combined with the carrier materials to produce a single dosage form will vary, depending such as the type of the condition and the subject to be treated. In general, a therapeutic composition contains from about 5% to about 95% active ingredient (w/w). More specifically, a therapeutic composition contains from about 20% (w/w) to about 80% or about 30% to about 70% active ingredient (w/w).

The peptides of the present invention can be formulated according to known methods for preparing pharmaceutically useful compositions. Formulations are described in detail in a number of sources which are well known and readily available to those skilled in the art. For example, Remington's Pharmaceutical Science by E. W. Martin describes formulations which can be used in connection with the subject invention. In general, the compositions of the subject invention will be formulated such that an effective amount of the bioactive compound(s) is combined with a suitable carrier in order to facilitate effective administration of the composition.

The preparation of a pharmacological composition that contains active ingredients dissolved or dispersed therein is well understood in the art and need not be limited based on formulation. Typically such compositions are prepared as injectables either as liquid solutions or suspensions; however, solid forms suitable for solution, or suspensions, in liquid prior to use also can be prepared. The preparation also can be emulsified, such as oil-in-water emulsion.

The active ingredient can be mixed with excipients which are pharmaceutically acceptable and compatible with the active ingredient and in amounts suitable for use in the therapeutic methods described herein. Suitable excipients are, for example, water, saline, glycerol, ethanol, sucrose, glucose, mannitol, sorbitol or the like and combinations thereof. In addition, if desired, the composition can contain minor amounts of auxiliary substances such as wetting or emulsifying agents, pH buffering agents and the like which enhance the effectiveness of the active ingredient.

Liquid compositions also can contain liquid phases in addition to and to the exclusion of water. Exemplary of such additional liquid phases are glycerin, vegetable oils such as cottonseed oil, and water-oil emulsions.

The invention also provides a pharmaceutical pack or kit comprising one or more containers filled with one or more of the ingredients, e.g., compound, carrier suitable for administration.

### Routes of Administration

The peptides and compositions of the present invention can be administered to the subject being treated by standard routes, including the oral, topical, transdermal, intra-articular, parenteral (e.g., intravenous, intraperitoneal, intradermal, subcutaneous or intramuscular), intracranial, intracerebral, intraspinal, intravaginal, intrauterine, or rectal route. Depending on the condition being treated, one route may be preferred over others, which can be determined by those skilled in the art.

The peptides of the present invention may also be administered utilizing liposome technology, slow release capsules, implantable pumps, and biodegradable containers. These delivery methods can, advantageously, provide a uniform dosage over an extended period of time. The amount of the therapeutic composition of the invention which is effective in the treatment of a particular disease, condition or disorder will depend on the nature of the disease, condition or disorder and can be determined by standard clinical techniques.

The dosage of effective amount of the peptides varies from and also depends upon the age and condition of each individual patient to be treated. In general, suitable unit dosages may be between about 0.01 to about 500 mg, about 0.01 to about 400 mg, about 0.01 to about 300 mg, about 0.01 to about 200 mg, about 0.01 to about 100 mg, or about 0.01 to about 50 mg. Such a unit dose may be administered once to several times (e.g. two, three or four times) every week, twice a week, or every day, according to the judgment of the practitioner and each patient's circumstances.

In addition, in vitro assays may optionally be employed to help identify optimal dosage ranges. The precise dose to be employed in the formulation will also depend on the route of administration, and the seriousness of the disease, condition or disorder, and should be decided according to the judgment of the practitioner and each patient's circumstances. Effective doses may be extrapolated from dose-response curves derived from in vitro or animal model test systems.

Illustratively, dosage levels of the administered active ingredients can be: intravenous, 0.01 to about 20 mg/kg (preferably 0.01 to about 1 mg/kg); intraperitoneal, 0.01 to about 100 mg/kg (preferably 0.01 to about 5 mg/kg); subcutaneous, 0.01 to about 100 mg/kg (preferably 0.01 to about 5 mg/kg); intramuscular, 0.01 to about 100 mg/kg (preferably 0.01 to about 5 mg/kg); orally 0.01 to about 200 mg/kg (more preferably about 1 to about 5 mg/kg) and preferably about 1 to 100 mg/kg (more preferably about 1 to about 5 mg/kg); intranasal instillation, 0.01 to about 20 mg/kg (preferably 0.01 to about 1 mg/kg); and aerosol, 0.01 to about 20 mg/kg (preferably 0.01 to about 1 mg/kg) of animal (body) weight.

The precise dose to be employed in the formulation will also depend on the route of administration, and the seriousness of the disease, condition, or disorder, and should be decided according to the judgment of the practitioner and each patient's circumstances. Effective doses may be extrapolated from dose-response curves derived from in vitro or animal model test systems, and such extrapolation of toxicological and pharmacological data from animals to humans is well within the knowledge of one skilled in the art. For example, in order to obtain an effective mg/kg dose for humans based on data generated from rat studies, the effective mg/kg dosage in rats may be divided by six. In certain embodiments, the effective dose administered to humans is at least about 5-fold lower, about 10-fold lower, about 20-fold lower, about 30-fold lower, or about 50-fold lower, than the effective dose administered to rats.

More specifically, a therapeutically effective dosage for treatment of stroke may depend on the seriousness of the disease condition, such as the volume of cerebral infarction. A higher dosage may be required for a subject with larger cerebral infarction. For instance, 4 mg/kg every 2 h×4 injections, i.p., of DADLE is therapeutically effective for rat subjects with 81.2±5.3 mm³ of infarcted striatal tissue, which corresponds roughly to ⅓ of one hemisphere of the rat brain (Examples 1-4). Similar approximations of this dosage to cerebral infarction volume can be used in humans. Additionally, a smaller volume of cerebral infarction may require a lower dosage, and a larger volume of cerebral infarction can require a higher dosage.

Further, the rat MCAor stroke model is an art-recognized model in the study of stroke and for prediction of pharmacological effects of a therapeutic agent for treating stroke in humans [38, 39, 40]. In addition, experimental conditions in the present rat MCAor stroke model are carefully adjusted according to pathological conditions of stroke and/or cerebral ischemic injury in actual clinical settings. For instance, the volume of cerebral infarction normally varies from one patient to another in actual clinical settings. As a result, the volume of cerebral infarction in the present MCAor rodent model is also varied among subjects, for accurate prediction of the therapeutic effects of the present delta opioid peptides.

In one embodiment, the peptides of the present invention and any second neuroprotective agent are administered sequentially to the patient, with the second neuroprotective agent being administered before, after, or both before and after treatment with peptides of the present invention. Sequential administration involves treatment with the second neuroprotective agent at least on the same day (within 24 hours) of treatment with peptides of the present invention and may involve continued treatment with the second neuroprotective agent on days that the peptides of the present invention is not administered.

### Materials and Methods

### Subjects

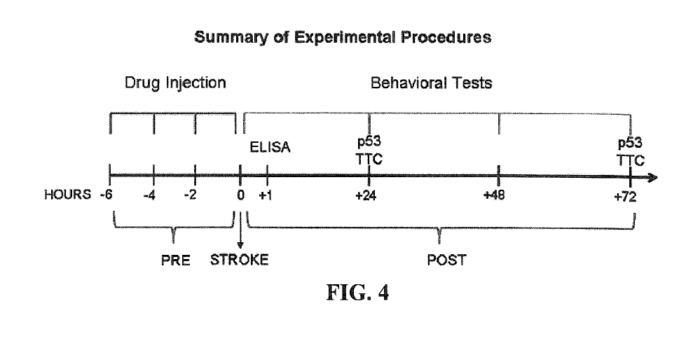

A total of 56 male Sprague-Dawley rats (Charles River, Ind.), weighing between 290-330 g, served as subjects in the present study. The animals were housed in a temperature-controlled room with normal 12-12 hr light-dark cycle. Food and water were freely available in the house cage. All animal handling and surgical procedures adhered to NIH IACUC guidelines. All investigators directly involved in drug treatments, behavioral testing and immunohistochemical analyses were blinded to the treatment conditions. A schematic diagram is provided summarizing experimental procedures of the present invention (FIG. 4).

### Treatment Conditions

To establish the protective property of DADLE in the central nervous system, young adult Sprague-Dawley rats were pretreated with DADLE (4 mg/kg every 2 hr×4 injections, i.p.) or saline prior to unilateral MCAor. To elucidate the opioid mediation on the effects of DADLE, some ischemic animals were treated with naltrexone (a universal opioid receptor blocker) or naloxone methiodide (a peripheral opioid receptor blocker). Animals were randomly assigned to one of the following treatment conditions: DADLE alone (n=10), DADLE+naltrexone (n=8), DADLE+naloxone methiodide (n=8), naloxone methiodide alone (n=8), naltrexone alone (n=8), saline vehicle alone (n=8), and normal, no surgery/no treatment (n=6). The present dosage of DADLE was within the reported efficacious range for protection against loss of dopamine transporter induced by chronic methamphetamine [13,14]. The dosages for naltrexone (0.1 mg/kg, i.p.) and naloxone methiodide (0.01 mg/kg, i.p.) were based on previous studies showing each drug's maximal antagonistic effects [30,31]. Each delta opioid antagonist was injected simultaneously with DADLE.

### Ischemia Surgery

Following the last drug injection, animals were subjected to MCAor surgery. The surgical procedures were done under aseptic conditions. Throughout the surgery and until recovery, body temperature was kept at 37° C. using a thermal blanket connected to a rectal probe that controlled the heat delivery to the animal. The experiments were performed using the MCAor embolic techniques as described in Borlongan et al., Exp Neurol 1998, 149:310-321 [17].

Specifically, under deep anesthesia using chloral hydrate (400 mg/kg, i.p.), the right common carotid artery was identified and isolated through a ventral midline cervical incision. A suture filament with its tip coated with a combination of dental substance mixture was used to allow not only a smooth finish (avoiding artery perforations during insertion into the lumen), but also, more importantly, a customized tip tapered to the desired gauge depending on the animal weight, age, and gender. Such highly customized filament tip blocks the MCA better than a flamed tip, allowing animals to develop better and consistent stroke and thus development of infarct within 1 hour faster than conventional flame-tapered filaments requiring 90 or 120 minutes of occlusion. Furthermore, TTC and laser Doppler measurements revealed that the present 1-hour MCAo produces infarct size and regional cerebral blood flow reduction (>80%) comparable with that achieved by 90- or 120-minute MCA occlusion using standard procedures. About 15 to 17 mm of the suture filament was inserted from the junction of the external and internal carotid arteries to block the MCA. The right MCA was occluded for one hr. Based on our own studies and several others, a one-hr occlusion of the MCA results in maximal infarction [17]. A major factor that could have led to the present infarcts limited to the striatum is the use of liquid anesthesia, as opposed to gas anesthesia. Since all animals received the same anesthesia, any differences in stroke outcome parameters across could be ascribed to the experimental drug.

### Behavioral Tests

After recovery from anesthesia, animals were evaluated on a battery of tests including spontaneous rotational test, elevated body swing test (EBST), postural bias test and forelimb akinesia test. It is reported by the present inventors that animals with successful MCA occlusion exhibit asymmetric behaviors [17]. Ischemic animals have been observed to rotate >2 full 360 degree ipsiversive turns, swing=75% towards the ischemic side, exhibit a postural deficit characterized by a clipped (to the chest) left forelimb and stretched-out right forelimb, and display an akinetic left forelimb [17].

The EBST is described in detail in Borlongan et al., Exp Neurol 1998, 149:310-321[17], and has been utilized to characterize the biased swing activity of ischemic animals starting at one month post-ischemia and extending up to three months post-surgery [17]. The animal was lifted 20 times by its tail and the direction of the swing was recorded. An ipsiversive swing activity of=75% has been suggested as reflective of successful unilateral ischemia or brain insult [17].

The spontaneous rotational test [32] was performed immediately after the animal's recovery from anesthesia following insertion of the embolus. The animal was placed in a chamber made of transparent Plexiglas (40×40×35.5 cm) and the direction of the animal's rotation was noted over two 5-minute sessions. Two full turns (tight ipsiversive rotations) per minute was considered indicative of a brain insult [17,32]. For the postural bias test and forelimb akinesia test, a semiquantitative scale was used as described in Bederson et al., Stroke 1986, 17:472-476 [33].

The postural tail-hang test involved holding the animal by the tail (similar to the EBST) and noting the positions of the forelimbs. A scale is used for grading the ischemic injury-induced dysfunctions as follows: 0, rats extend straight both forelimbs, no observable deficit; 1, rats keep the left forelimb to the breast and extend straight the right forelimb; 2, rats show decreased resistance to lateral push in addition to behavior in score 1 without circling; 3, rats twist the upper half of their body in addition to behavior in score 2.

Finally, the forelimb akinesia test involved pulling each forelimb at 90 degrees away from the body median axis. The return of the forelimb towards the midline was scored as 0 for slow movement with rigidity, 1 for slow movement, and 2 for smooth rapid movement. A score of 1 in the scale for postural bias test or forelimb akinesia test was considered as indicative of CNS dysfunction [17]. All of the tests were used to characterize any ameliorative effects of DADLE on ischemia-induced dysfunctions from day 0 to day 3 after MCAor surgery.

### Cerebral Infarction Assay

The volume of infarction was analyzed during a histological assay using triphenyltetrazolium chloride (TTC) for determining dehydrogenase activity. Animals were euthanized at either 24 hr or 72 hr after MCAor surgery. Under deep anesthesia (chloral hydrate, 500 mg/kg, i.p.) animals were perfused intracardially with saline. Details of the TTC procedure were described in Wang et al., J Neurosci 1997, 17:4341-4348 [6]. The volume of infarction was measured in each slice and summed using computerized planimetry (PC-based Image Tools software). The volume of infarction was computed as: 2 mm (thickness of the slice)×[sum of the infarction area in all brain slices (mm²)] [6]. To minimize artifacts produced by post-ischemic edema in the infarcted area, the infarction area in the ipsilateral hemisphere was indirectly measured by subtracting the noninfarcted area in the ipsilateral hemisphere from the total intact area of the contralateral hemisphere. An additional cohort of ischemic animals (n=4) underwent the same treatment paradigm and were euthanized at 72 hours post-stroke for propidium iodide (P-3566, Molecular Probes, 20 mg/kg via tail vein) staining to further characterize necrotic cell death.

Determination of p53 mRNA Expression

To examine whether DADLE altered programmed cell death or apoptosis, which has been postulated to mediate ischemia-induced cell injury [7,8], the mRNA expression of the apoptotic marker p53 were assayed. At 24 hr and 72 hr after MCAor surgery, randomly selected animals that received either DADLE alone or saline alone were euthanized by rapid decapitation. The striatum was dissected and quickly frozen using dry ice then stored in −80° C. freezer until tissue processing was conducted. Total RNA extraction, northern blot analysis and hybridization were conducted using standard methods as described in Chomczynski et al., Anal Biochem 1987, 162:156-159 and Mattson, Brain Res 2000, 886:47-53 [34,35]. Analyses of resulting bands were quantified using a Macintosh computer-based image analysis system (Image, NIH). Densitometrically determined intensities of p-53 mRNA was normalized to 18S rRNA.

### Immunohistochemistry

In order to further characterize the effects of DADLE on apoptotic cell death, 20 animals were subjected to the same procedures as above, and the treatment effects of DADLE (n=10) against saline vehicle alone (n=10) were compared. Rats were euthanized at 72 hours after MCAor surgery. The rats were perfused transcardially with 200 ml of cold phosphate-buffered saline (PBS) and 200 ml of 4% paraformaldehyde in PBS. Brains were removed and post-fixed in the same fixative overnight at 4° C. with the subsequent replacement with 30% sucrose in PBS for 72 hours. The brains were coronally sectioned at the thickness of 8 μm. Sections were washed 3 times for 5 minutes in PBS. Sections were then incubated overnight at 4° C. with caspase-3 (Abeam, 1:500) or Fas (Santa Cruz Biotechnology, 1:100) primary antibody and washed 3 times in PBS. Afterwards, sections were incubated with corresponding Cy3-conjugated secondary antibodies (1:1000, Jackson ImmunoResearch Lab, PA) for 90 minutes. Finally, sections were washed 3 times for 5 minutes each in distilled water, and cover-slipped with Gelmount (Biomedia Corp., Foster City, Calif.). Control studies included exclusion of primary antibody substituted with 10% normal horse serum in PBS. No immunoreactivity was observed in these controls.

For morphological analyses, immunoreactive cells in the striatum within the ipsilateral to the stroke hemisphere were examined using a Zeiss LSM510 confocal microscope (Oberkochen, Germany). Specifically, 6 coronal sections at every 300 μm that approximately captured the ischemic striatum (AP −2.0 to +2.0 mm from the bregma) were examined from each rat and the number of positive cells was counted in each 6 high power fields and the averages were used for the statistical analyses. Alternate sections from the additional cohort of animals used for propidium iodide (see cerebral infarction assay above) were processed for caspase-3 immunofluorescent imaging to further reveal apoptotic cell death.

### ELISA Assay

To examine possible non-opioid effects of DADLE, assays of expression of neurotrophic factors (glial cell line-derived neurotrophic factor (GDNF) and nerve growth factor (NGF)) that have been previously shown to be protective against ischemia were performed [6,36]. Enzyme-linked immunosorbent assay (ELISA) was conducted on ischemic animals injected either with DADLE or saline (n=6 per group). Following the same drug dosing regimen mentioned above, animals were rapidly decapitated, their brains removed and striatal and frontal cortical tissues quickly dissected. Brain homogenization and supernatant acidification were performed using methods as described in Okragly et al., Exp Neurol 1997, 145:592-596 [37] with minor modifications. Protein concentrations were measured by using the BCA Kit (Pierse, Rockford, Ill. USA). For the measurement of GDNF, mouse monoclonal anti-GDNF antibody (R & D Systems, Inc., Minnesota, USA) was used as a capture antibody and biotinylated goat anti-GDNF antibody (R & D Systems, Inc., Minnesota, USA) was used as a detection antibody. For the measurement of NGF, the NGF Emax TM ImmunoAssay system (Promega Cooperation, Madison, Wis., USA) was used. The THERMOmax 96-well microplate reader (Molecular Devices Corp., Sunnyvale, Calif. USA) was used to measure the optical densities.

### Statistical Analyses

Analysis of variance (ANOVA) followed by posthoc test using Fisher's protected least significant difference (PLSD) was used to reveal statistical significance in behavioral, mRNA, ELISA, and histological data. A statistically significant difference was pre-set at p<0.05.

Following are examples that illustrate procedures for practicing the invention. These examples should not be construed as limiting. All percentages are by weight and all solvent mixture proportions are by volume unless otherwise noted.

### EXAMPLES

### Example 1

### Attenuation of Motor Asymmetrical Behaviors by DADLE

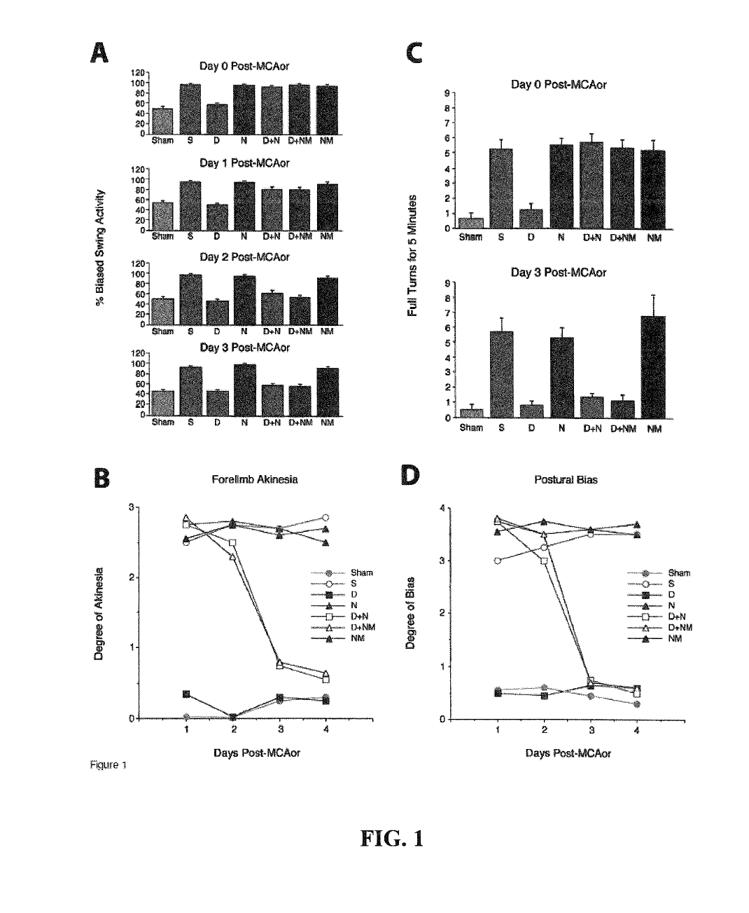

This Example demonstrates that DADLE attenuates motor asymmetrical behaviors. Daily behavioral tests revealed that ischemic animals treated with DADLE displayed near-normal behaviors throughout the post-MCAor test period in a battery of tests (FIG. 1). In contrast, ischemic animals pretreated with saline, naltrexone alone, or naloxone methiodide alone displayed significant abnormalities in EBST, spontaneous rotational test, postural bias test, and forelimb akinesia test throughout the post-MCAor test period. The mean locomotor deficits in these ischemic animals are as follows: 82.8%±12.3 (mean percentage biased swing response), 2.52±0.56 (mean ipsiversive rotations per minute), 2.25±0.31 (mean postural bias score), and 1.17±0.15 (mean akinesia score). Ischemic animals pretreated with DADLE+naloxone methiodide or DADLE+naltrexone also exhibited similar behavioral deficits at 24 hr post-MCAor, but showed near-normal behaviors at 48 hr and 72 hr post-MCAor. This observation of transient behavioral abnormalities in ischemic animals treated with DADLE+opioid antagonists suggest that DADLE only partially exerted its protection via the opioid system, and non-opioid mechanisms appear to mediate the majority of protective effects of DADLE.

### Example 2

### Reduction of Cerebral Infarction by DADLE

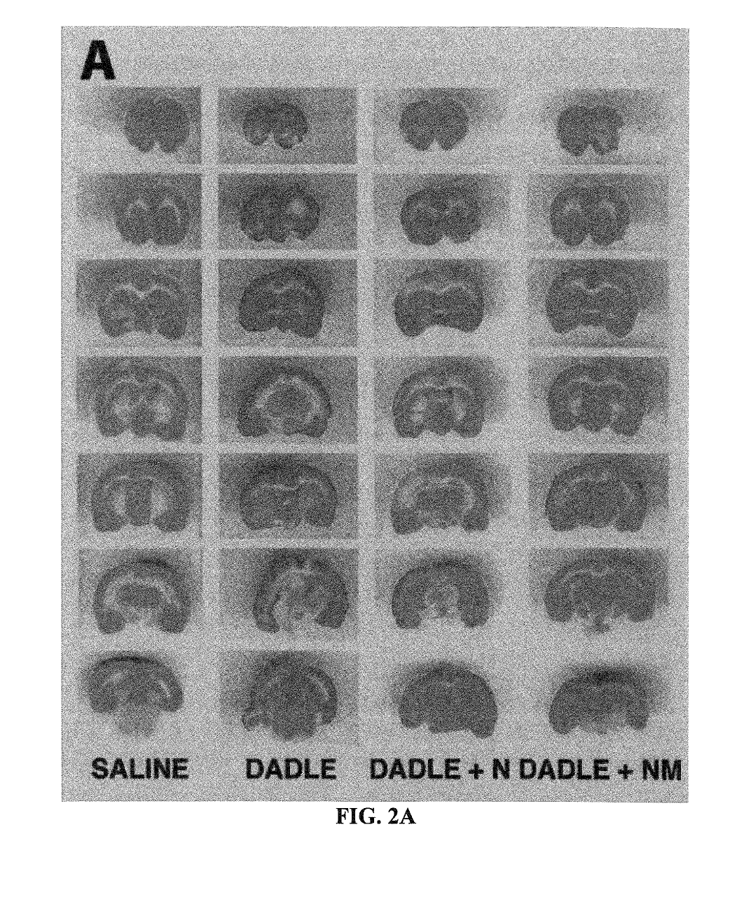

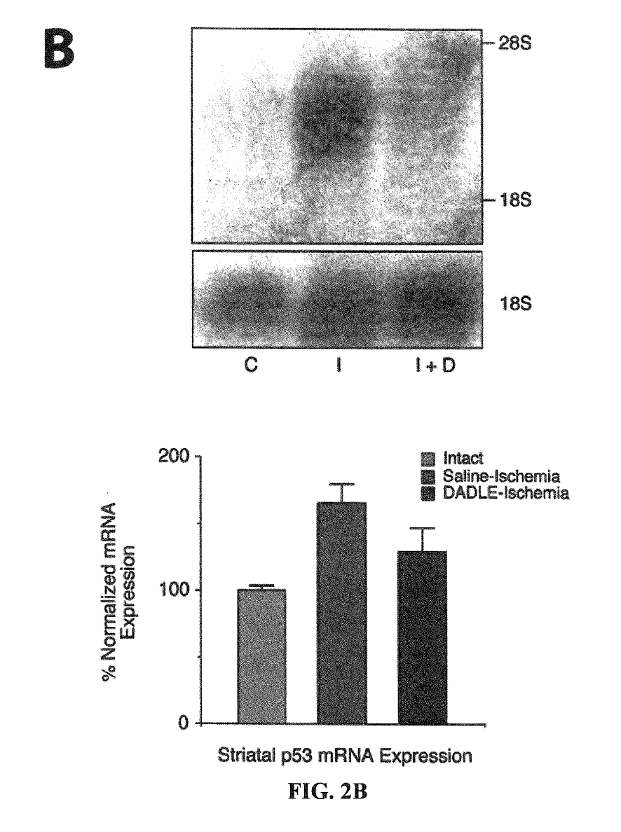

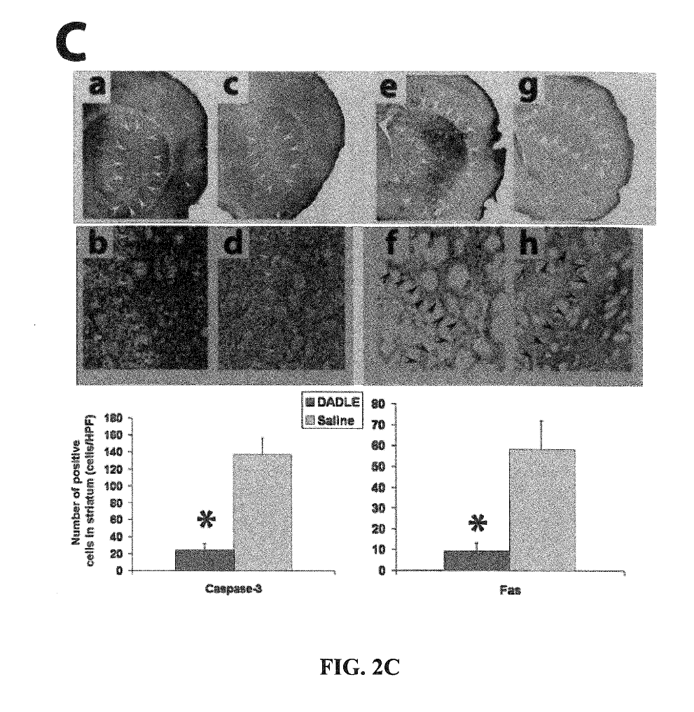

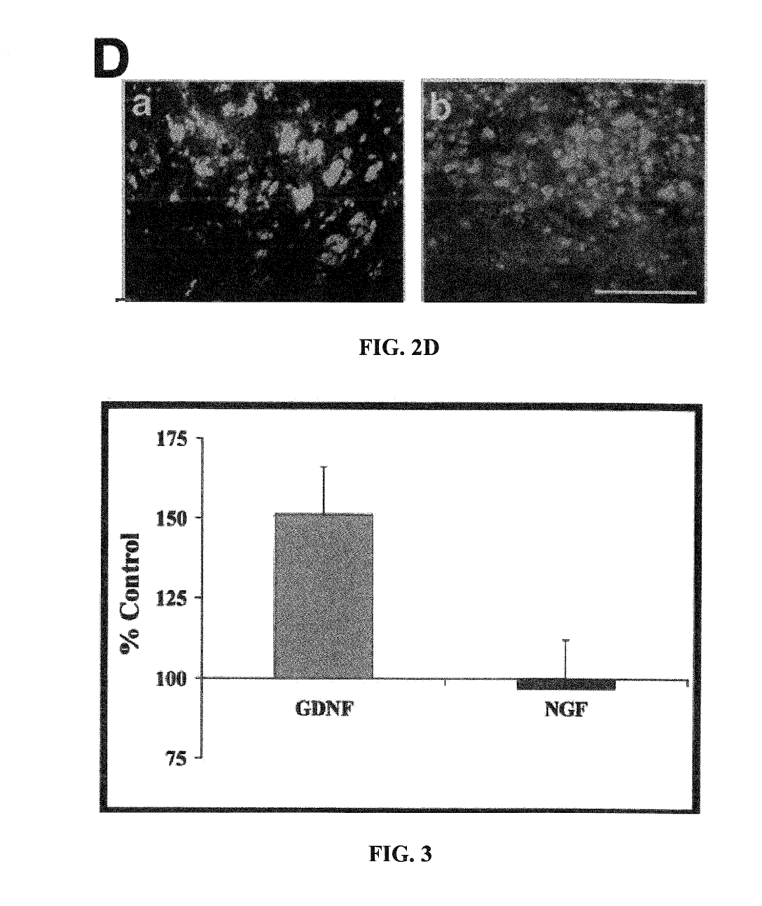

This Example demonstrates that DADLE reduces cerebral infarction and protects against necrotic cell death associated with ischemia-reperfusion injury. Triphenyltetrazolium chloride (TTC) staining at 24 or 72 hr after reperfusion revealed that brains from ischemic animals that were treated with DADLE, alone or with adjuvant opioid blockers, had almost completely intact striata, whereas those from ischemic animals that received saline showed significant infarction in the lateral striatum (FIG. 2A). Ischemic animals pretreated with saline had a mean volume (±S.E.M.) of 81.2±5.3 mm³ of infarcted striatal tissue, while ischemic animals pretreated with DADLE alone, DADLE+naltrexone or DADLE+naloxone methiodide had no detectable infarction. Ischemic animals that received naltrexone alone or naloxone methiodide alone had a mean volume of 78.7±7.4 mm³ of striatal infarcted tissue, which did not differ from that of the ischemic animals received saline alone. ANOVA revealed significant treatment effects (p<0.0001) and posthoc t-tests revealed significant reduction in infarct volume in DADLE-treated ischemic animals, including those co-administered with opioid blockers, compared to saline-treated ischemic animals (p's<0.05). These histological observations were consistent for both time periods of histological examination. The observation of almost intact striatum following MCAor in DADLE-treated animals, even with the co-administration of opioid antagonists, suggests that DADLE protected against necrotic cell death processes induced by ischemia-reperfusion injury.

### Example 3

### Decrease of Ischemia Induced-Apoptotic Cell Death by DADLE

Analyses of apoptotic cell death revealed a significant increment in the mRNA expression of p-53 in the striatum of ischemic animals that received saline, while those that received DADLE exhibited near-normal striatal p-53 expression. NOVA revealed a significant difference across treatment conditions (F2,15=5.6, p<0.05) (FIG. 2B). Normalized values of p-53 mRNA expression showed that vehicle-treated ischemic animals had a significant increase in mRNA expression of p-53 in the ischemic striatum (65% increment) at 24 hr post-MCAor surgery compared to the intact striatum of control, normal animals (p<0.01). In contrast, there was no significant difference in p-53 mRNA expression between the ischemic striatum (28% increment) of DADLE-treated ischemic animals and the intact striatum of control, normal animals (p>0.05). Comparisons of ischemic striata showed a marked reduction (but only a trend, p=0.07) in p-53 mRNA expression in DADLE-treated ischemic animals compared to vehicle-treated ischemic animals. Moreover, immunohistochemical analyses of phenotypic markers of apoptosis revealed significant reductions in caspase-3- and Fas-positive cells in DADLE-treated ischemic animals compared to vehicle-treated ischemic animals (FIG. 2C). These results indicate that DADLE protected against apoptotic cell death processes associated with ischemia-reperfusion injury.

### Example 4

### Increase of Levels of GDNF Expression by DADLE

ELISA examination of expression of neurotrophic factors revealed elevated levels of GDNF, but not NGF, in the striatal and cortical tissues harvested from ischemic animals treated with DADLE (FIG. 3). Ischemic animals treated with DADLE had significantly higher levels of GDNF compared to those treated with saline (p<0.05). However, both groups did not differ in their levels of NGF (p>0.05). These results indicate that DADLE specifically increased GDNF, but not NGF.

All patents, patent applications, provisional applications, and publications referred to or cited herein are incorporated by reference in their entirety, including all figures and tables, to the extent they are not inconsistent with the explicit teachings of this specification.

The terms “a” and “an” and “the” and similar referents as used in the context of describing the invention are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein. Unless otherwise stated, all exact values provided herein are representative of corresponding approximate values (e.g., all exact exemplary values provided with respect to a particular factor or measurement can be considered to also provide a corresponding approximate measurement, modified by “about,” where appropriate).

The use of any and all examples, or exemplary language (e.g., “such as”) provided herein, is intended merely to better illuminate the invention and does not pose a limitation on the scope of the invention unless otherwise indicated. No language in the specification should be construed as indicating any element is essential to the practice of the invention unless as much is explicitly stated.

The description herein of any aspect or embodiment of the invention using terms such as “comprising”, “having”, “including” or “containing” with reference to an element or elements is intended to provide support for a similar aspect or embodiment of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

It should be understood that the examples and embodiments described herein are for illustrative purposes only and that various modifications or changes in light thereof will be suggested to persons skilled in the art and are to be included within the spirit and purview of this application.

### REFERENCES

1. Maldonado R, Saiardi A, Valverde O, Samad T A, Rogues B P, Borrelli E: Absence of opiate rewarding effects in mice lacking dopamine D2 receptors. Nature 1997, 388:586-589.
2. Cook C D, Rodefer J S, Picker M J: Selective attenuation of the antinociceptive effects of mu opioids by the putative dopamine D3 agonist 7-OH-DPAT. Psychopharmacology 1999, 144:239-247.
3. Boutin H, Dauphin F, MacKenzie E T, Jauzac P: Differential time-course decreases in nonselective, mu-, delta-, and kappa-opioid receptors after focal cerebral ischemia in mice. Stroke 1999, 30:1271-1277.
4. Frerichs K U, Hallenbeck J M: Hibernation in ground squirrels induces state and species-specific tolerance to hypoxia and aglycemia: an in vitro study in hippocampal slices. J Cereb Blood Flow Metab 1998, 18:168-175.
5. Kevelaitis E, Peynet J, Mouas C, Launay J M, Menasche P: Opening of potassium channels: the common cardioprotective link between preconditioning and natural hibernation? Circulation 1999, 99:3079-3085.
6. Wang Y, Lin S Z, Chiou A L, Williams L R, Hoffer B J: Glial cell line-derived neurotrophic factor protects against ischemia-induced injury in the cerebral cortex. J Neurosci 1997, 17:4341-4348.
7. Hayashi T, Sakai K, Sasaki C, Itoyama Y, Abe K: Loss of bag-1 immunoreactivity in rat brain after transient middle cerebral artery occlusion. Brain Res 2000, 852:496-500.
8. Wang Y, Chang C F, Morales M, Chou J, Chen H L, Chiang Y H, Lin S Z, Cadet J L, Deng X, Wang J Y, Chen S Y, Kaplan P L, Hoffer B J: Bone morphogenetic protein-6 reduces ischemia-induced brain damage in rats. Stroke 2001, 32:2170-2178.
9. Yin D, Mufson R A, Wang R, Shi Y: Fas-mediated cell death promoted by opioids. Nature 1999, 397: 218.
10. Shibata S, Tominaga K, Watanabe S: kappa-Opioid receptor agonist protects against ischemic reduction of 2-deoxyglucose uptake in morphine-tolerant rats. Eur J Pharmacol 1995, 279:197-202.
11. Woodburn V L, Oles R, Poat J, Woodruff G N, Hughes J: Anticonvulsant agents, dizocilpine maleate, enadoline and HA 966 have different effects on N-methyl-DL-aspartate-induced immediate early gene induction in mice. Neuroscience 1993, 56:703-709.
12. Hayashi T, Tsao L I, Cadet J L, Su T P: D-Ala2, D-Leu5]enkephalin blocks the methamphetamine-induced c-fos mRNA increase in mouse striatum. Eur J Pharmacol 1999, 366:7-8.
13. Tsao L, Cadet J L, Su T P: Reversal by [D-Ala2,D-Leu5]enkephalin of the dopamine transporter loss caused by methamphetamine. Eur J Pharmacol 1999, 372:5-7.
14. Tsao L, Ladenheim B, Andrews A M, Chiueh C C, Cadet J L, Su T P: Delta opioid peptide [D-Ala2,D-leu5]enkephalin blocks the long-term loss of dopamine transporters induced by multiple administrations of methamphetamine: involvement of opioid receptors and reactive oxygen species. J Pharmacol Exp Ther 1998, 287:322-331.
15. Butterfield D A, Koppal T, Howard B, Subramaniam R, Hall N, hensley K, Yatin S, Allen K, Aksenov M, Aksenova M, Carney J: Structural and functional changes in proteins induced by free radical-mediated oxidative stress and protective action of the antioxidants N-tert-butyl-alpha-phenylnitrone and vitamin E. Ann N Y Acad Sci 1998, 854:448-462.
16. Fujimura M, Morita-Fujimura Y, Kawase M, Copin J C, Calagui B, Epstein C J, Chan P H: Manganese superoxide dismutase mediates the early release of mitochondrial cytochrome C and subsequent DNA fragmentation after permanent focal cerebral ischemia in mice. J Neurosci 1999, 19:3414-3422.
17. Borlongan C V, Tajima Y, Trojanowski J Q, Lee V M, Sanberg P R: Transplantation of cryopreserved human embryonal carcinoma-derived neurons (NT2N cells) promotes functional recovery in ischemic rats. Exp Neurol 1998, 149:310-321.
18. Onizuka K, Fukuda A, Kunimatsu M, Kumazaki M, Sasaki M, Takaku A, Nishino H: Early cytopathic features in rat ischemia model and reconstruction by neural graft. Exp Neurol 1996, 137:324-332.
19. Nishino H, Czurko A, Onizuka K, Fukuda A, Hida H, Ungsuparkorn C, Kunimatsu M, Sasaki M, Karadi Z, Lenard L: Neuronal damage following transient cerebral ischemia and its restoration by neural transplant. Neurobiology 1994, 2:223-234.
20. Borlongan C V, Zhou F C, Hayashi T, Su T P, Hoffer B J, Wang Y: Involvement of GDNF in neuronal protection against 6-OHDA-induced parkinsonism following intracerebral transplantation of fetal kidney tissues in adult rats. Neurobiol Dis 2001, 8:636-646.
21. Lin L F, Doherty D H, Lile J D, Bektesh S, Collins F: GDNF: a glial cell line-derived neurotrophic factor for midbrain dopaminergic neurons. Science 1993, 260:1130-1132.
22. Borlongan C V, Su T P, Wang Y: Treatment with delta opioid peptide enhances in vitro and in vivo survival of rat dopaminergic neurons. Neuroreport 2000, 11:923-926.
23. Borlongan C V, Su T P, Wang Y: Delta opioid peptide augments functional effects and intrastriatal graft survival of rat fetal ventral mesencephalic cells. Cell Transplant 2001, 10:53-58.
24. Paratcha G, Ibáñez C F, Ledda F: GDNF is a chemoattractant factor for neuronal precursor cells in the rostral migratory stream. Mol Cell Neurosci 2006, 31:505-514.
25. Kobayashi T, Ahlenius H, Thored P. Kobayashi R, Kokaia Z, Lindvall O: Intracerebral infusion of glial cell line-derived neurotrophic factor promotes striatal neurogenesis after stroke in adult rats. Stroke 2006, 37:2361-2367.
26. Stenman E, Jamali R, Henriksson M, Maddahi A, Edvinsson L: Cooperative effect of angiotensin AT(1) and endothelin ET(A) receptor antagonism limits the brain damage after ischemic stroke in rat. Eur J Pharmacol 2007, 570:142-148.
27. Hossmann K A: Pathophysiology and therapy of experimental stroke. Cell Mol Neurobiol 2006, 26:1057-1083.
28. Kearns C M, Cass W A, Smoot K, Kryscio R, Gash D M: GDNF protection against 6-OHDA: time dependence and requirement for protein synthesis. J Neurosci 1997, 17:7111-7118.
29. Fox C M, Gash D M, Smoot M K, Cass W A: Neuroprotective effects of GDNF against 6-OHDA in young and aged rats. Brain Res 2001, 896:56-63.
30. Frost J J, Wagner Jr H N, Dannals R F, Ravert H T, Links J M, Wilson A A, Burns H D, Wong D F, McPherson R W, Rosenbaum A E: Imaging opiate receptors in the human brain by positron tomography. J Comput Assist Tomogr 1985, 9:231-236.
31. Shacoori V, Saiag B, Rault B: Melatonin modifies prolactin release induced by opiate antagonists in male rats. Endocr Res 1995, 21:545-553.
32. Nikkhah G, Cunningham M G, McKay R, Bjorklund A: Dopaminergic microtransplants into the substantia nigra of neonatal rats with bilateral 6-OHDA lesions. II, Transplant-induced behavioral recovery. J Neurosci 1995, 15:3562-3570.
33. Bederson J B, Pitts L H, Tsuji M, Nishimura M C, Davis R L, Bartkowiski H M: Rat middle cerebral artery occlusion: evaluation of the model and development of a neurologic examination. Stroke 1986, 17:472-476.
34. Bienz B, Zakut-Houri R, Givol D, Oren M: Analysis of the gene coding for the murine cellular tumour antigen p53. EMBO J 1984, 3:2179-2183.
35. Chomczynski P, Sacchi N: Single-step method of RNA isolation by acid guanidinium thiocyanate-phenol-chloroform extraction. Anal Biochem 1987, 162:156-159.
36. Mattson M P: Neuroprotective signaling and the aging brain: take away my food and let me run. Brain Res 2000, 886:47-53.
37. Okragly A J, Haak-Frendscho M: An acid-treatment method for the enhanced detection of GDNF in biological samples. Exp Neurol 1997, 145:592-596.
38. Chopp M, Steinberg G K, Kondziolka D, Lu M, Bliss T M, Li Y, Hess D C, Borlongan C V: Who's in favor of translational cell therapy for stroke: STEPS forward please?. Cell Transplant. 2009; 18(7):691-3.
39. Stem Cell Therapies as an Emerging Paradigm in Stroke Participants., Stem Cell Therapies as an Emerging Paradigm in Stroke (STEPS): bridging basic and clinical science for cellular and neurogenic factor therapy in treating stroke. Stroke., 2009 February; 40(2):510-5.
40. Borlongan C V, Chopp M, Steinberg G K, Bliss T M, Li Y, Lu M, Hess D C, Kondziolka D. Potential of stem/progenitor cells in treating stroke: the missing steps in translating cell therapy from laboratory to clinic. Regen Med. 2008 May; 3(3):249-50.

What is claimed is:

1. A method of promoting neurogenesis in a human subject, wherein said method comprises administering to a human subject in need of such treatment an effective amount of an isolated peptide of SEQ ID NO:1 or salt thereof, whereby neurogenesis is promoted.

2. The method according to claim 1, wherein the salt is selected from the group consisting of hydrochloric salt, acetic salt, aspartic salt, citric salt, fumaric salt, hippuric salt, lactic salt, malic salt, phosphoric salt, sulfuric salt, succinic salt, carbonic salt, and gluconic salt.

3. The method according to claim 1, wherein the subject is diagnosed with stroke.

4. The method according to claim 3, used to treat ischemic stroke.

5. The method according to claim 3, used to treat cerebral artery stroke.

6. The method according to claim 3, used to delay progression of stroke.

7. The method according to claim 1, used to treat ischemic injury or cerebral infarction.

8. The method according to claim 1, used to treat a motor deficit selected from the group consisting of atypical movement, bradykinesia, postural bias or instability, impaired balance or coordination, muscular rigidity, and ataxia.

9. The method according to claim 1, used to reduce neuronal cell apoptosis, necrosis, or both.

10. The method according to claim 1, wherein said isolated peptide or salt thereof is administered to the subject at a dose of about 0.01 to about 5 mg/kg per unit dose.

11. A method of increasing glial cell-derived neurotrophic factor (GDNF) expression in a human subject, wherein said method comprises administering to the subject an effective amount of an isolated peptide of SEQ ID NO:1 or salt thereof, whereby GDNF expression is increased.

12. The method according to claim 11, wherein the salt is selected from the group consisting of hydrochloric salt, acetic salt, aspartic salt, citric salt, fumaric salt, hippuric salt, lactic salt, malic salt, phosphoric salt, sulfuric salt, succinic salt, carbonic salt, and gluconic salt.

13. The method according to claim 11, wherein the subject is diagnosed with stroke.

14. The method according to claim 13, used to treat ischemic stroke.

15. The method according to claim 13, used to treat cerebral artery stroke.

16. The method according to claim 11, wherein said isolated peptide or salt thereof is administered to the subject at a dose of about 0.01 to about 5 mg/kg per unit dose.


SEQUENCE LISTING (1 sequence)

SEQ ID NO: 1 - protein, 5 aa, Artificial
  delta opioid peptide
YAGFL